(19) United States
(12) Patent Application Publication
(10) Pub. No.: US 2021/0295730 A1
(43) Pub. Date: Sep. 23, 2021

(54) SYSTEM AND METHOD FOR VIRTUAL REALITY MOCK MRI

(71) Applicants: Max OROZCO, Los Angeles, CA (US); Noah OROZCO, Los Angeles, CA (US)

(72) Inventors: Max OROZCO, Los Angeles, CA (US); Noah OROZCO, Los Angeles, CA (US)

(21) Appl. No.: 17/204,917

(22) Filed: Mar. 17, 2021

Related U.S. Application Data
(60) Provisional application No. 62/991,520, filed on Mar. 18, 2020.

(51) Int. Cl. G09B 9/00; G06F 3/01; G06T 13/40; G06T 13/80; G09B 19/00; A61B 5/00

(52) U.S. Cl. CPC G09B 9/00; G06F 3/012; A61B 5/6803; G06T 13/80; G09B 19/00; G06T 13/40

(57) ABSTRACT

A method and system configured to use a virtual reality headset to generate a dynamic MRI simulation for assessment and training purposes. The system can use a predictive analytic model to analyze and assess a wide range of biometric data for predicting the user's suitability for the actual MRI. The prediction can be displayed via a built-in indicator on the headset. The system can provide real-time biofeedback training to improve the user's performance in the actual MRI.

### CROSS-REFERENCE TO RELATED APPLICATION

This application claims priority to U.S. Proviosnal Application No. 62/991,520, entitled “SYSTEM AND METHOD FOR VIRTUAL REALITY MOCK MRI” filed on Mar. 18, 2020, the content of which is expressly incorporated by reference herein in its entirety.

### TECHNICAL FIELD

The disclosure generally relates to a system and method for mock Magnetic Resonance Imaging (MRI) using virtual reality technology.

### BACKGROUND

Magnetic Resonance Imaging (MRI) is a common and important diagnostic tool for medical practitioners, researchers, and patients. However, MRI procedures can be daunting for children and some adults as it is loud and conducted in a narrowly enclosed area. Furthermore, participants or users need to lie still during the procedure to provide diagnostic-quality images. As a result, a large number of children and adults with difficulty remaining still are sedated for the imaging procedure. To reduce the cost of sedation, lower sedation risks, and to improve the quality of the MRI images, mock MRI simulators have been used to train participants or users in a controlled environment.

### SUMMARY

In some implementations, a virtual reality headset is configured to provide a simulation of an actual MRI scanning or a mock MRI. The simulation provides an immersive experience of the actual MRI scanning, including the visual and audio aspects. During the simulation, the virtual reality headset can receive, via biometric sensors, multi-source biosensing raw data and create digital biometrics data of the user in real-time. The created digital biometric data can further comprise user biometric scores reflecting the user's age, gender, movement, stress, alertness, emotional valence. According to some embodiments, a composite user score can be created based on various digital biometric data.

In some implementations, using a predictive analytic model, the virtual reality headset can analyze the user's biometrics data and/or user scores and predict the user's suitability for an actual MRI without sedation. According to some embodiments, one or more user composite scores based on a range of different digital biometric data, e.g., an emotional valance score, a heart rate score, can be calculated and used as the inputs for the predictive analytic model. In some implementations, the predictive analytic model is based on an MRI database comprising age-normed biometric datasets. The prediction is generated by assessing the user's digital biometric data and/or user scores in relation to the predictive analytic model. In some embodiments, the predictive analytic model can use different data mining techniques to process and analyze the user's biometric data and/or user scores in real-time.

In some implementations, the virtual reality headset is configured to generate a prediction of the user's suitability for the actual MRI without sedation based on the assessment as described herein. The prediction can be one of a pass, a failure, and a recommendation of repeating the mock MRI. According to some embodiments, the prediction is displayed via a built-in light or indicator on the headset. In other embodiments, the prediction can be displayed on a user interface of a separate computing device in communication with the headset. According to some embodiments, the virtual reality headset can also recommend the user to repeat the mock MRI to improve his/her performance.

In some implementations, the virtual reality headset is configured to provide real-time biofeedback training for improving the participant's or user's performance in an actual MRI. According to some embodiments, the virtual reality headset is configured to show real-time outputs of the related biometric sensors via audio and visual cues. As a result, the participant can control or adjust his/her body to meet the demands of the simulated MRI.

In some implementations, a method of using a virtual reality headset for a mock MRI is disclosed. The method comprises: generating, via the virtual reality headset, a simulation of an actual MRI to a user, wherein the simulation comprises the audio and visual experiences of the actual MRI, receiving multi-source raw data and creating digital biometric data such as biometric scores of the user over a predetermined period of time, assessing the user's suitability for the actual MRI without sedation by analyzing the digital biometric data, e.g., biometric scores, in a predictive analytic model, and generating a prediction of the user's suitability for the actual MRI. The simulation can further display a target image configured to gauge the participant's movement.

In some implementations, another method of using a virtual reality headset for a mock MRI is disclosed. The method comprises: generating, via the virtual reality headset, a simulation of an actual MRI to a user, displaying a target image in the simulation, receiving multi-source biosensing raw data via a plurality of biometric sensors in communication with the virtual reality headset over a predetermined period of time, creating digital biometric data of the user, evaluating the digital biometric data and/or user scores in relation to a predictive analytic model for the user's suitability in an actual MRI, determining that the user's digital biometric data and/or user scores exceeds at least one predetermined threshold, and displaying real-time biofeedback associated with the target image, wherein the real-time biofeedback comprises a visual and/or an audio reminder.

In some implementations, a virtual reality headset system for a mock MRI comprises: a processor, one or more biometric sensors, and memory storing instructions that, when executed by the processor, cause the virtual reality headset system to generate a simulation of an actual MRI to a user, display a target image in the simulation, create biometric data of the user via collected raw data from one or more biometric sensors, by assessing the biometric data and/or user scores in a predictive analytic model, evaluate the user's suitability for the actual MRI, and generate a prediction of the user's suitability for the actual MRI.

Details of one or more implementations are set forth in the accompanying drawings and the description below. Other features, aspects, and potential advantages will be apparent from the description and drawings, and from the claims.

### DESCRIPTION OF DRAWINGS

FIG. 1 is an example flow diagram illustrating a method of a mock MRI, according to some embodiments;

FIG. 2 is another example flow diagram illustrating another method of a mock MRI, according to some embodiments;

FIG. 3 is a perspective view of a virtual reality headset configured for providing the mock MRI, according to some embodiments;

FIGS. 4A, 4B, and 4C are an exemplary view of a user using the virtual reality headset embedded with location and rotation sensors for the mock MRI, according to some embodiments;

FIG. 5 is an exemplary view of a user using the virtual reality headset in communication with a plurality of biosensors for the mock MRI and an exemplary flow diagram, according to some embodiments;

FIG. 6 is an exemplary image of a mock MRI, according to some embodiments;

FIG. 7 is another exemplary image of the mock MRI, according to some embodiments;

FIG. 8 is another exemplary image of the mock MRI, according to some embodiments;

FIG. 9 is another exemplary image of the mock MRI, according to some embodiments;

FIG. 10 is another exemplary image of the mock MRI, according to some embodiments;

FIG. 11 is an exemplary first-person view of the mock MRI, according to some embodiments;

FIG. 12 is another exemplary first-person view of the mock MRI with a target image, according to some embodiments;

FIG. 13 is another exemplary first-person view of the mock MRI with real-time feedbacks, according to some embodiments;

FIG. 14 is another exemplary image of the mock MRI, according to some embodiments; and

FIG. 15 is a block diagram of basic components of a virtual reality headset implementing the features and processes of FIGS. 1-14.

### DETAILED DESCRIPTION

Various embodiments of the present technology are discussed in detail below. While specific implementations are discussed, it should be understood that this is done for illustration purposes only. A person skilled in the relevant art will recognize that other components and configurations may be used without departing from the spirit and scope of the present technology.

The MRI simulator has been used to train the MRI participants, particularly children, in preparation for the actual MRI scan. Traditionally, the mock MRI simulator is a replica of an MRI scanner without the magnetic field. As it is as big as the actual MRI scanner, the MRI simulator requires a simulation room of similar size to the radiology suite. The MRI simulator provides a realistic experience of the actual MRI procedure. However, the traditional MRI simulator is bulky and expensive, rendering it impractical when space or resource is limited. Thus, there is a need to provide an immersive training procedure to acclimate the participant to the actual scanning environment with a fraction of the cost and the required space.

Furthermore, the present clinical procedure calls for all children under a certain age, e.g., seven years old, to be sedated for the MRI scan. Given the high cost of sedation and inherent risks, there is a need to determine whether a participant, e.g., a child of a certain age, is capable of going through an MRI scan without sedation or anesthesia. The present subject matter discloses a method and system to evaluate whether a child or a user could go through the actual MRI without sedation by providing an immersive mock training that is efficient and easily accessible.

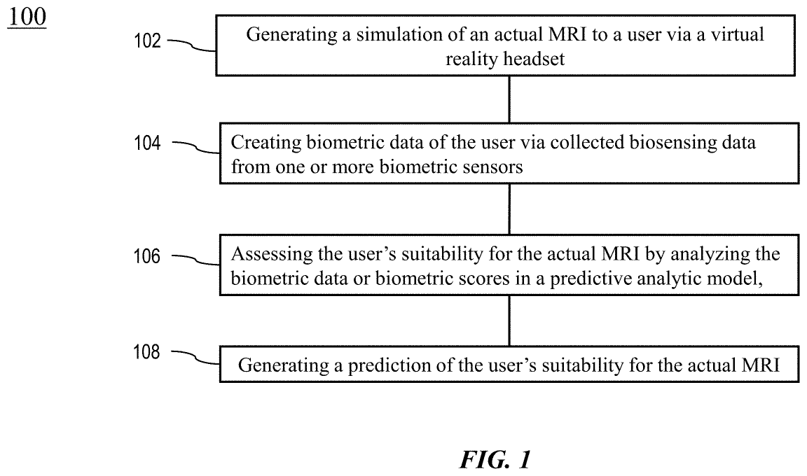

FIG. 1 is an example flow diagram illustrating a method of a mock MRI. According to some embodiments, a virtual reality headset, such as an Oculus headset, can be used to generate a mock MRI, as illustrated by the flow diagram 100.

At step 102, the virtual reality headset can generate an immersive simulation of an actual MRI process to a participant or user via the head-mounted display and headphones. By simulating both the visionary and acoustic aspects of the actual MRI, the virtual reality headset enables the user to acclimate to scanning. It is well known that the actual MRI scan makes a loud sound as metal coils vibrate by rapid pulses of electricity. The scanning sound can reach as high as 110 decibels of noise, with variations in pitch and intensity. Accordingly, the headphone or speaker of the headset can generate a vibrating sound equivalent to the actual scanning sound in the sound level, pitch, and intensity.

According to some embodiments, the immersive simulation rendered by the headset can be interactive and dynamic. For example, it can show the shape and dimension of the inner MRI bore in a dynamic view. According to some embodiments, the dynamic inner view of the MRI bore can be adjusted according to the viewing angle of the user or the speed of the user's movement.

At step 104, the virtual reality headset can create biometric data of the user via collected biosensing data from one or more biometric sensors in communication with the virtual reality headset over a predetermined period of time. According to some embodiments, the plurality of biometric sensors can be built-in sensors of the headset, e.g., cameras, accelerometers. According to some embodiments, additional external biometric sensors can be used to create the biometric data in the same manner as described herein.

Examples of the biometric sensors, for example, include cameras, accelerometers, magnetometers, gyroscopes, blood pressure meters, pulse sensors, respiration sensors, heart rate sensors, eye-tracking sensors, EDA (electrodermal activity) sensors, EMG (Electromyography) sensors, EEG (Electroencephalography) sensors, ECG (Electrocardiogram) sensors, and galvanic skin response sensors. The biometric sensors can convert received physical signals to digital biometrics data. Furthermore, a person skilled in the relevant art can recognize that other sensors may be used in compliance with the spirit of the present subject matter.

According to some embodiments, during the mock MRI, a wide range of raw biosensing data collected from biometric sensors can be collected and analyzed in real-time. According to some embodiments, the virtual headset or a computing device in communication with the headset can create one or more user biometric data via various digital signal processing methods. Examples of the user biometric data include user biometric scores to reflect the user's physical and emotional state in response to the MRI simulation, such as the user's level of anxiety, stress, state of arousal, emotional valence and motion capabilities, etc. For example, the user's anxiety and stress level can be reflected by the respiration sensor data or the blood pressure data, and the user's state of arousal can be reflected by the galvanic skin sensor data. According to some embodiments, the digital biometric data or score can reflect the user's head movement, eye movement, body movement, heart rate, galvanic skin response, facial expression, muscle tension, posture, respiration during the mock MRI. According to some embodiments, the headset can generate a composite user score based on various individual sensor data, wherein individual sensor data can be assigned with different weight in the composite score.

According to some embodiments, the user's known static biometric data such as age and gender can be added and evaluated in combination with the dynamically measured digital biometric data. For example, the system can receive input about the user's age and/or gender before commencing the mock MRI.

According to some embodiments, the predetermined period of time is the whole length of the mock MRI. In addition, the predetermined period of time can be the partial length of the mock MRI, for example, 3-5 minutes.

At step 106, the virtual reality headset can assess the user's suitability for the actual MRI by analyzing the digital biometric data such as user biometric scores in a predictive analytic model. According to some embodiments, the predictive analytic model can comprise an MRI database based on datasets from a plurality of reference users. According to some embodiments, the MRI database can comprise age-normed movement datasets. For example, the movement datasets from reference users at different ages, e.g., 5-year-old user, or 8-year-old user, are grouped and collectively analyzed. According to some embodiments, the MRI datasets can comprise age-normed digital biometric datasets that comprise a full range of biometric data such as user's age, gender, movement, stress, alertness, emotional valence.

According to some embodiments, the predictive analytic model is configured to incorporate various digital biometric data or biometric scores as input parameters. Such digital biometric data comprises known biometric data and dynamic digital biometric data provided by the biometric sensors in real-time. According to some embodiments, the predictive analytic model is configured to evaluate the relationship of these digital biometrics or biometric scores as compared to existing datasets in the MRI database using different data mining and machine learning techniques. Examples of such techniques include, for example, data mining and pattern isolation, data cleaning and preparation, pattern prediction, classification, data clustering, multiple linear regression, random forest, decision tree, prediction, etc. Accordingly, the user's actual MRI performance can be predicted by assessing the user's digital biometric data in the MRI simulation in relation to the predictive analytic model.

According to some embodiments, the headset can generate a target image within the simulated MRI interior. An example of the target image is a bullseye-type image that is used to gauge the head or body movement of the user. The target image can indicate a permitted threshold of movement. According to some embodiments, any movement exceeding a permitted threshold, e.g., 2 mm in any direction with regard to the target image, can be counted as a breach. According to some embodiments, any movement beyond a predetermined frequency, e.g., five times in 1 minute, can be counted as a breach. Upon detection of a breach, a visual breaching cue can be displayed next to the target image to remind the user to keep still. According to some embodiment, an audio breaching cue, e.g., a beep, can be provided in conjunction with the visual cue.

At step 108, the virtual reality headset can generate a prediction of the user's suitability for the actual MRI without sedation, based on the assessment as described herein. The prediction estimates the likelihood of a user to successfully take the actual MRI without sedation and produce qualifying images for the diagnosis purpose. The prediction can be one of a pass, a failure, and a recommendation of repeating the mock MRI. A pass means the user is recommended to take the actual MRI without sedation. A failure means the user is recommended to be sedated for taking the actual MRI. According to some embodiments, the prediction can be an estimation of likelihood, e.g., 80%, for a successful MRI without sedation.

According to some embodiments, the prediction can be shown via a built-in light or an indicator on the headset. Different light combinations can be configured to show different recommendations. For example, a green light can indicate that the perspective MRI user is expected to be capable of taking the actual MRI without sedation. Red light can indicate that the user is recommended to take the MRI with sedation. Furthermore, a flashing red light can suggest an immediate termination of the mock MRI due to the user's evident unsuitability or discomfort. In addition, yellow light can indicate a recommendation of repeating the mock MRI to improve the user's chance of success. According to some embodiments, when the participant receives a yellow light in the first mock MRI, the user is recommended to take a second or more mock MRI until he/she is familiar with the procedure.

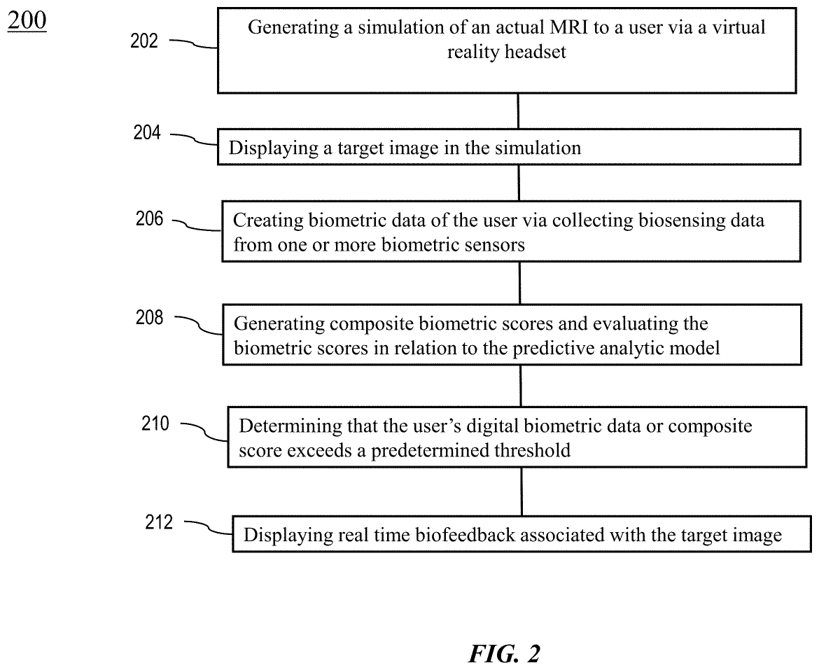

FIG. 2 is another example flow diagram illustrating another method of a mock MRI. At step 202, a virtual reality headset can generate a mock MRI as illustrated by the flow diagram 200. According to some embodiments, prior to the mock MRI, the simulation can comprise an introduction video of the MRI process, such as how it works and why it is important to remain still. For a young user such as a five-year-old child, an animated avatar can be shown in the video to explain the process. According to some embodiments, the young user can review and select a favorite avatar among a list of available avatars.

At step 204, the virtual reality headset can generate a target image within the simulated MRI interior. An example of the target image is a bullseye-type image for gauging the user's movement. The target image can indicate a permitted threshold of movement and reflect the movement of the user. Another example of the target image is the selected avatar, e.g., a cartoon teddy bear.

At step 206, the virtual reality headset can create biometric data of the user via collected raw biosensing data via a plurality of biometric sensors over a predetermined period of time. According to some embodiments, the plurality of biometric sensors can be built-in sensors of the headset, e.g., cameras, accelerometers. According to some embodiments, additional external biometric sensors can be utilized to collect the digital biometric data in the same manner as described herein.

According to some embodiments, during the mock MRI, the virtual reality headset or a computing device in communication with it can create a wide range of digital biometric data from the collected raw sensor data in real-time. The user biometric data can reflect the user's physical and emotional state in response to the MRI simulation, such as the user's level of anxiety, stress, state of arousal, emotional valence and motion capabilities, etc. For example, the user's anxiety and stress level can be reflected by the respiration sensor data or the blood pressure data, and the user's state of arousal can be reflected by the galvanic skin sensor data. According to some embodiments, the digital biometric data can be user scores to reflect the user's head movement, eye movement, body movement, heart rate, galvanic skin response, facial expression, muscle tension, posture, respiration during the mock MRI.

According to some embodiments, the biometric sensors can collect comprehensive motion data related to the user's body movement, such as the frequency and degree of the user's head movement. According to some embodiments, in addition to the head movement, various movements, such as torso movement, arm, and leg movement, can be collected. Examples of appliable biometers or biometric sensors, for example, include optical sensors such as cameras and non-optical sensors such as accelerometers, magnetometers, gyroscopes.

According to some embodiments, the biometric sensors such as blood pressure meters, heart rate sensors, eye-tracking sensors, EEG sensors, ECG sensors, and galvanic skin response sensors can collect other biometric data, including heart rate, galvanic skin response, eye movement, facial expression, muscle tension, posture, respiration, and electroencephalogram (EEG) to create digital biometrics indicative of anxiety, stress, state of arousal, emotional valence and motion capabilities, etc. for a comprehensive assessment of a user's suitability for the actual MRI.

According to some embodiments, the user's known biometric data such as age and gender can be considered in combination with the dynamically measured digital biometric data. For example, the system can receive input about the user's age and/or the user's gender before commencing the mock MRI. According to some embodiments, the predetermined period of time is the whole length of the mock MRI or partial of the mock MRI.

During the mock MRI, the collected data can be transmitted to the headset via, for example, the Bluetooth protocol. According to some embodiments, the collected data to the headset via other wired or wireless communication protocols.

At step 208, the virtual reality headset can generate composite biometric scores based on a plurality of types of biometric data and evaluate the composite scores in relation to the predictive analytic model. According to some embodiments, the predictive analytic model can comprise an MRI database based on datasets from a plurality of reference users. According to some embodiments, the MRI database can comprise age-normed movement datasets. For example, the movement datasets are collected and analyzed by reference users at different ages, e.g., the 5-year-old user or the 8-year-old user. According to some embodiments, the MRI datasets can comprise age-normed digital biometric datasets that comprise a full range of biometric data such as user's age, gender, movement, stress, alertness, emotional valence.

According to some embodiments, the predictive analytic model is configured to incorporate various digital biometric data as input parameters. Such digital biometric data comprises known biometric data and real-time digital biometric data provided by the biometric sensors. According to some embodiments, the predictive analytic model is configured to incorporate the user composite scores as input parameters. According to some embodiments, the predictive analytic model is configured to evaluate the relationship of real-time biometrics or the user composite scores as compared to existing datasets in the MRI database using different data mining and processing techniques. Examples of such techniques include, for example, data mining and pattern isolation, data cleaning and preparation, pattern isolation, classification, data clustering, multiple linear regression, random forest, decision tree, prediction, etc. Accordingly, the user's actual MRI performance can be predicted by assessing the user's digital biometric data in the MRI simulation in relation to the predictive analytic model.

According to some embodiments, parameters for the predictive analytical model comprises digital biometric data and/or user composite scores reflecting the user's physical and emotional state, such as the user's age, gender, the user's level of anxiety, stress, state of arousal, emotional valence and motion capabilities. According to some embodiments, a motion capability co-efficient is developed by the summation of breaches of the reference users.

At step 210, the virtual reality headset can determine that at least part of the user's digital biometric data or composite scores exceeds at least one predetermined threshold. According to some embodiments, the virtual reality headset can determine that at least one of the value related to the user's heart rate, galvanic skin response, facial expression, muscle tension, posture, respiration exceeds at least one predetermined threshold or a preferred range. For example, the adult user's heart rate is measured at more than 100 beats per minute. For example, the virtual reality headset can determine that the user's movement frequency and/or range is higher than a predetermined range and can consider it as a breach. For example, the user's head movement or body movement has exceeded a permitted range, e.g., 2 cm, in any direction with regard to the target image. Similarly, the user's frequency of movement is higher than a predetermined level, e.g., five times per minute.

At step 212, the virtual reality headset can display real-time biofeedback associated with the target image to reinforce motion awareness. The real-time biofeedback can comprise visual or audio breaching cues. According to some embodiments, the virtual reality headset can provide real-time training for improving the user's performance in an actual MRI. According to some embodiments, the virtual reality headset is configured to show real-time outputs of the related biometric sensors via audio and visual cues. As a result, the user can control or adjust his/her body to meet the demands of the simulated MRI. For example, during a mock MRI, by receiving and analyzing the outputs of the biometric sensor, after determining that the user's head movement or body movement has exceeded a permitted range, the virtual reality headset can display a visual reminder, e.g., “stay still” on the headset display.

According to some embodiments, the predictive analytic model is configured to analyze the user's digital biometric data or user composite scores throughout the VR simulation. According to some embodiments, the predictive analytic model can be created by collecting and analyzing user digital biometric data in relation to existing MRI datasets. According to some embodiments, the MRI datasets are age normed digital biometric datasets. Accordingly, the predictive analytic model can predict a user's biometric performance in the actual MRI based on biometric performance in the VR simulation.

At step 210, when the movement exceeds the permitted threshold, e.g., the movement that meets or exceeds 2 mm in roll (x-axis), pitch (y-axis), and/or yaw (z-axis), and/or exceeds a predetermined range in the three degrees of translation, a breaching cue can be shown to the user. For example, when the movement exceeds a predetermined frequency or degree, a visual breaching cue can be displayed next to the target image to remind the user to keep still. According to some embodiment, an audio breaching cue, e.g., a beep, can be provided in conjunction with the visual cue. At least according to some embodiments, the size or strength of the breaching cue can be variable depending on the degree or frequency of the detected movement.

According to some embodiments, the virtual reality headset can provide live training for improving the user's performance in an actual MRI. According to some embodiments, the virtual reality headset is configured to show real-time outputs of the related biometric sensors via audio and visual cues. As a result, the user can control or adjust his/her body to meet the demands of the simulated MRI. For example, during a mock MRI, by receiving and analyzing the outputs of the biometric sensor, the virtual reality headset can display a visual reminder, e.g., “stay still.”

At step 212, the virtual reality headset can generate a prediction of the user's suitability for the actual MRI based on the assessment as described herein. The prediction estimates the likelihood of a user taking the actual MRI without sedation. According to some embodiments, the prediction can be shown via a built-in light indicator on the headset. Different light combinations can be configured to show different recommendations. For example, a green light can indicate that the perspective MRI user is expected to be capable of taking the actual MRI without sedation. Red light can indicate that the user is recommended to take the MRI with sedation. Furthermore, a flashing red light can suggest an immediate termination of the mock MRI due to the user's evident unsuitability or discomfort. In addition, yellow light can indicate a recommendation of repeating the Mock MRI to improve the user's likelihood of success.

According to some embodiments, the prediction can be shown via an interface external to the headset. For example, the prediction can be transmitted to a computing device in communication with the headset, where it can be displayed via a user interface. Furthermore, other methods for displaying the prediction can be adopted without limiting to the methods described herein.

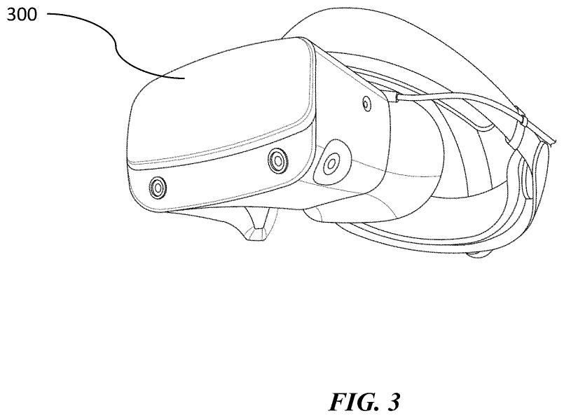

FIG. 3 is a perspective view of a virtual reality headset 300 configured to provide the mock MRI. An example of the virtual reality headset is an Oculus headset. Any other virtual reality headsets with similar functions, e.g., HTC headset, Google headset, can be used to implement the mock MRI. The virtual reality headset 300 can comprise a stereoscopic head-mounted display for displaying the mock MIR simulation. The virtual reality headset 300 can comprise speakers and/or headphones for generating the simulated vibrating sound of the mock MRI.

According to some embodiments, the virtual reality headset 300 can comprise head motion tracking sensors such as gyroscopes, accelerometers, magnetometers, etc. Additionally, the virtual reality headset 300 can comprise eye-tracking sensors and cameras. As described herein, during the mock MRI, these sensors can individually and collectively monitor and collect the user's physical and emotional state, such as the user's head movement, eye movement, body movement, heart rate, galvanic skin response, facial expression, muscle tension, posture, respiration, and an electroencephalogram.

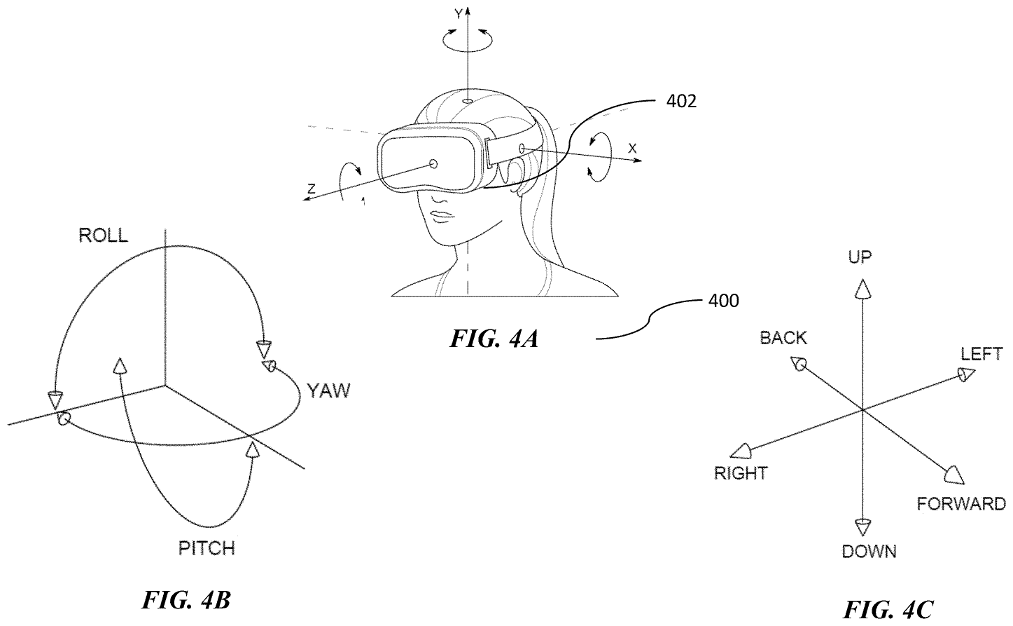

FIGS. 4A, 4B, and 4C are an exemplary view of a user 400 using the virtual reality headset 402 with embedded sensors for the mock MRI. As shown in FIG. 4A, the virtual reality headset 400 can measure motion and orientation in six degrees of freedom (6 DOF) with sensors such as accelerometers and gyroscopes.

As shown in FIG. 4A, the biometric sensors can receive both rotation and translation data at six degrees of freedom. As shown in FIG. 4B, according to some embodiments, the gyroscope can measure rotational data along the three-dimensional X-axis (pitch), Y-axis (yaw), and Z-axis (roll). As shown in FIG. 4C, according to some embodiments, the accelerometer can measure translational or motion data along the three-dimensional X-axis (forward-back), Y-axis(up-down), and Z-axis(right-left). The magnetometer can measure which direction the user 400 is facing.

According to some embodiments, the biometric sensors can collect motion and orientation data of the user's head motion, such as the frequency and degree of the user's head movement. When the head movement exceeds the permitted threshold, e.g., the head movement that meets or exceeds 2 mm in roll (x-axis), pitch (y-axis), and/or yaw (z-axis), a breaching cue can be shown to the user. For example, when the head movement exceeds a predetermined frequency or degree, a visual breaching cue can be displayed next to the target image to remind the user to keep still. According to some embodiment, an audio breaching cue, e.g., a beep, can be provided in conjunction with the visual cue. At least according to some embodiments, the size or strength of the breaching cue can be variable depending on the degree or frequency of the detected movement. According to some embodiments, in addition to the head motion, the biometric sensors can collect motion and orientation data of the user's body motion, such as torso or limbs motion.

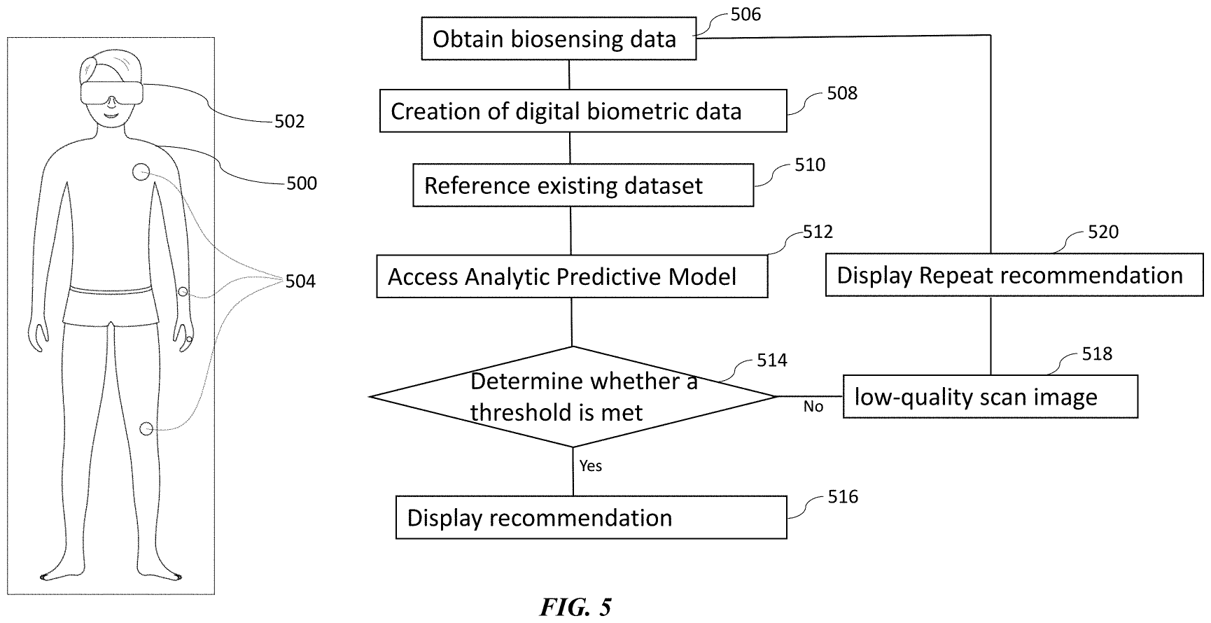

FIG. 5 is an exemplary view of a user 500 using the virtual reality headset 502 in communication with a plurality of biometric sensors 504 for the mock MRI according to the exemplary flow chart. As illustrated in FIG. 5, a user is lying on his back on a bed or a flat surface in a way similar to the user's posture on the actual MRI table.

According to some embodiments, the plurality of biometric sensors can be built-in sensors of the headset, e.g., cameras, accelerometers, or external biometric sensors such as blood pressure meters, pulse sensors, galvanic skin response sensors, etc. Other examples of the biometric sensors, for example, include cameras, accelerometers, magnetometers, gyroscopes, blood pressure meters, pulse sensors, respiration sensors, heart rate sensors, eye-tracking sensors, EDA (electrodermal activity) sensors, EMG (Electromyography) sensors, EEG (Electroencephalography) sensors, ECG (Electrocardiogram) sensors, and galvanic skin response sensors. The biometric sensors can convert received signals to biosensing data. Furthermore, a person skilled in the relevant art can recognize that other sensors may be used in compliance with the spirit of the present subject matter.

During the mock MRI, the collected multi-source biosensing raw data can be used to create digital biometric data via typical digital data processing methods. The created digital biometric data can be transmitted to the virtual reality headset in real-time via, for example, the Bluetooth protocol. According to some embodiments, other wired or wireless communication protocols can be employed. According to some embodiments, the real-time collected data can be transmitted to a computing unit in connection with the virtual reality headset.

At step 506, the headset 502 can display a simulated MRI to user 500. Also, headset 502 can obtain biosensing raw data from the biometric sensors 504. At step 508, headset 502 can create biometric data of the user via collected raw biosensing data. The headset 502 can generate composite biometric scores based on various types of biometric data. At step 510, headset 502 can evaluate the composite scores in reference to the existing datasets. For example, the existing datasets can be MRI databases based on datasets from a plurality of reference users. According to some embodiments, the MRI database can comprise age-normed movement datasets. According to some embodiments, the MRI datasets can comprise age-normed digital biometric datasets that comprise a full range of biometric data such as user's age, gender, movement, stress, alertness, emotional valence.

At step 512, headset 502 can access the predictive analytic model with the composite biometric scores in reference to the MRI database. According to some embodiments, the predictive analytic model is configured to incorporate the user composite scores as input parameters. According to some embodiments, the predictive analytic model is configured to evaluate the relationship of real-time biometrics or the user composite scores as compared to existing datasets in the MRI database using different data mining and processing techniques.

At step 514, headset 502 can determine whether user 500 has met a threshold for predicting to stay still for the actual MRI. When user 500 has met the threshold, at step 516, headset 502 can display a recommendation for the user to take the actual MIR without sedation. At step 518, headset 502 can determine that the user's composite scores have exceeded a preferred range. As a result, user 500 has failed to meet the threshold and is expected to receive low-quality scan images in the actual MRI. At step 520, headset 502 can display a recommendation for user 500 to repeat the mock MRI. As an alternative, headset 504 can display a recommendation for user 500 to take the mock MRI with sedation.

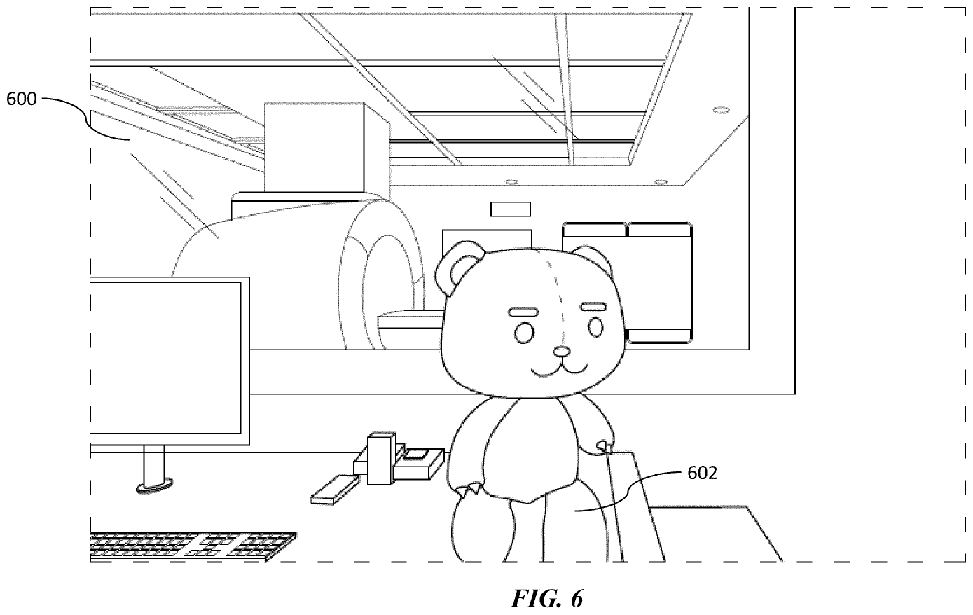

FIG. 6 is an exemplary image of a mock MRI that is generated by the virtual reality headset. The mock MRI can comprise an introduction video 600 that features an animated avatar 602 based on the age or preference of the user. For example, for users of three to seven years old, a teddy bear can explain how MRI works and what to expect during the scan. According to some embodiments, a list of different avatars is available, allowing the user to select a favorite character from the list.

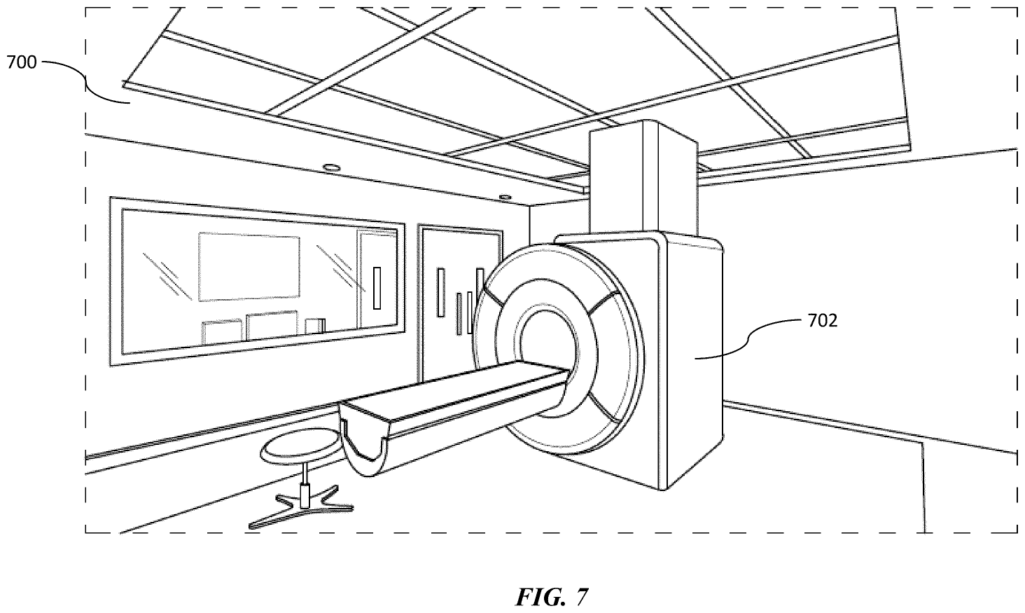

FIG. 7 is another exemplary image of the introduction video 700. It explains what the user is expected to see in the radiology suite that hosts the MRI scanner 702. As shown in FIG. 7, the view of the radiology suite can be interactive, three-dimensional, and immersive so that it helps the user to get familiar with the actual scanning environment.

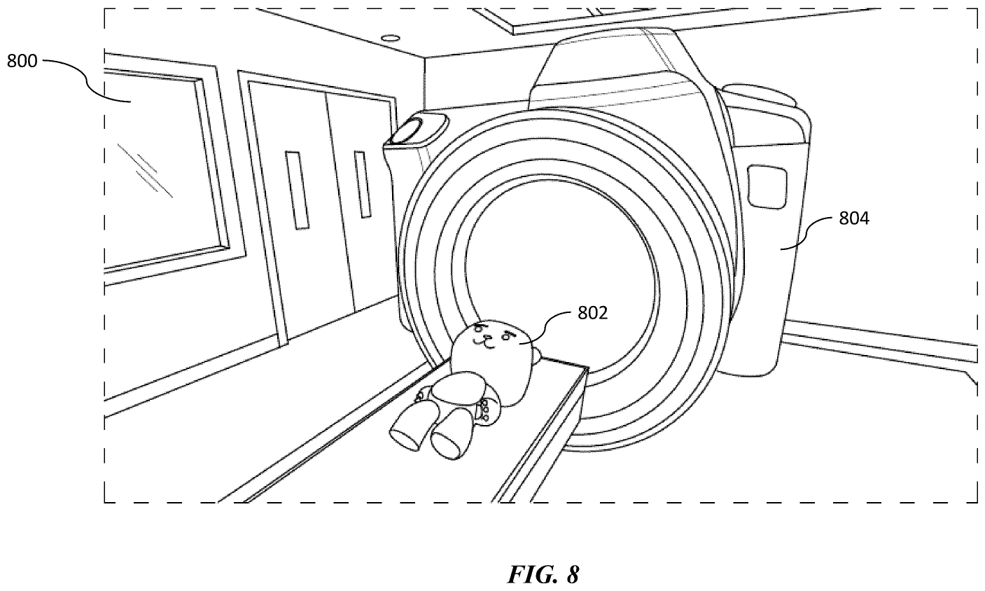

FIG. 8 is another exemplary image of the introduction video 800. For example, the avatar 802 can demonstrate how to lay flat on the scanning surface. The avatar 802 can further explain why it is important to keep still during the scanning. For example, it can compare the MRI scanner to a massive camera 804 with a huge lens for receiving and “take photos” of the user. Similar to taking a picture with a camera, the avatar 802 needs to stay still during the MRI scanning to avoid blurring the images.

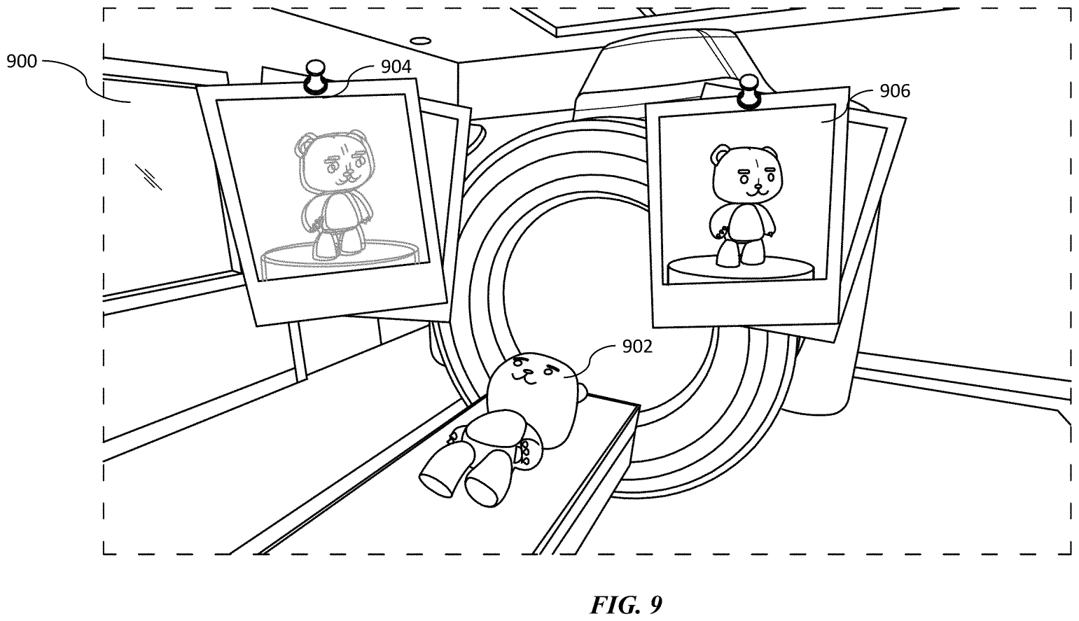

FIG. 9 is another exemplary image of the introduction video 900. For example, when the avatar 902 moves during the MRI, the resulting image 904 is blurred. By contrast, when the avatar 904 remains still, the resulting image 906 is clear and high quality, which explains why it is important to remain still during the scanning process.

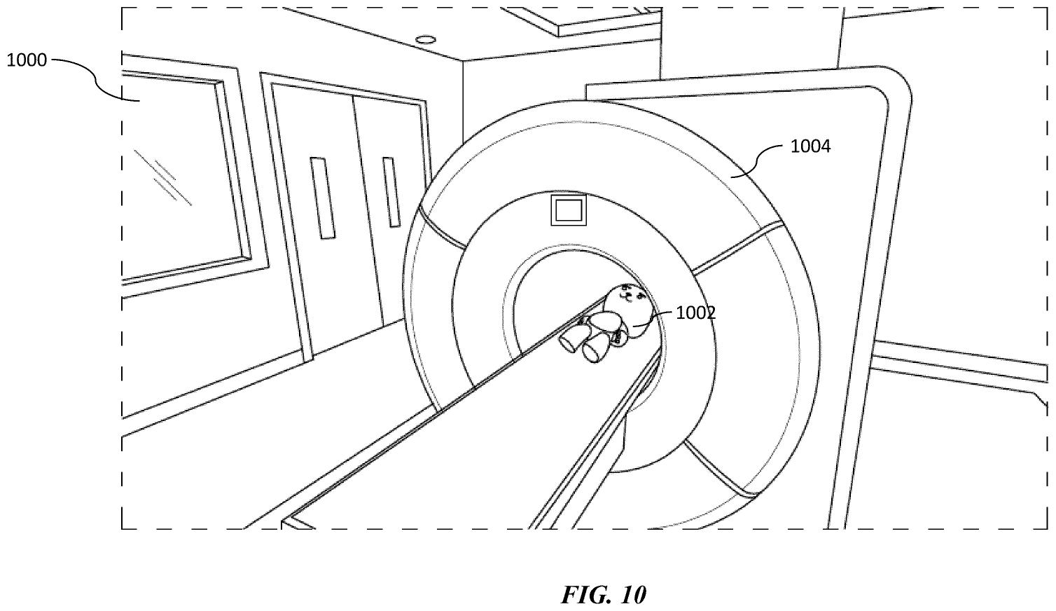

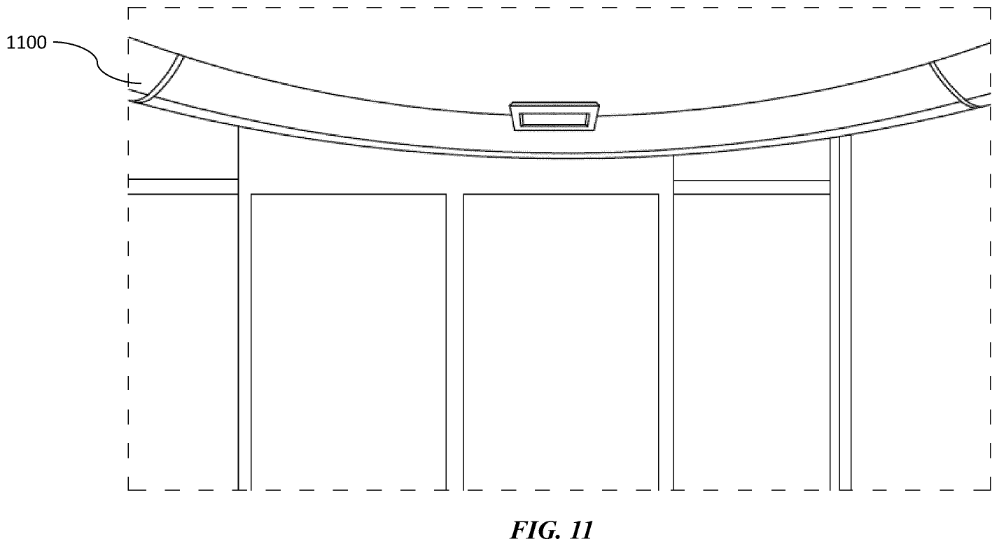

FIG. 10 is another exemplary image of the introduction video 1000. As shown in FIG. 10, the avatar 1002 is lying on the patient table in the bore of the MRI scanner 1004. FIG. 11 is an exemplary first-person view of the mock MRI 1100 while the user is in the MRI bore. The first-person view, for example, can simulate what the user will see while lying in the actual MRI bore. The three-dimensional image can be dynamic according to the movement of the user. For example, when the user tilts his or her head to the left side, the image accordingly adjusts to the left inner view of the bore wall. Besides the dynamic view, the user can hear a mock scanning sound that is produced by the headphone of the virtual reality headset. The mock scanning sound can be a loud noise equivalent to the actual MRI scanning noise in the decibel level, pitch, and frequency.

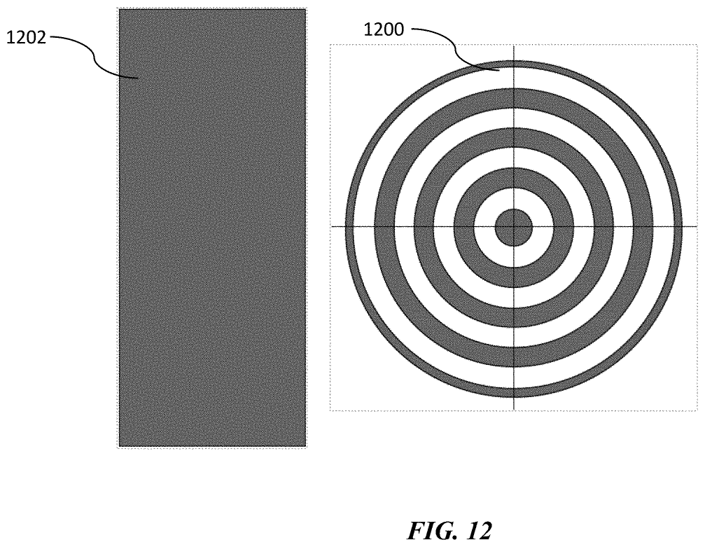

FIG. 12 is another exemplary first-person view of the mock MRI showing a target image 1200. The target image 1200 can be displayed over the mock MRI's interior view. An example of the target image is a bullseye-type image that is used to gauge the movement of the user.

According to some embodiments, the target image 1200 can reflect the head or body movement of the user. According to some embodiments, the virtual reality headset can provide real-time training for improving the user's performance in an actual MRI. When the user's movement exceeds a certain frequency or degree, a breaching cue 1202 can be generated by the headset to remind the user to remain still. According to some embodiment, the breaching cue 1202 can be a visual cue or an audio cue, or a combination of the two. As a result, the user can control or adjust his/her body to meet the demands of the simulated MRI.

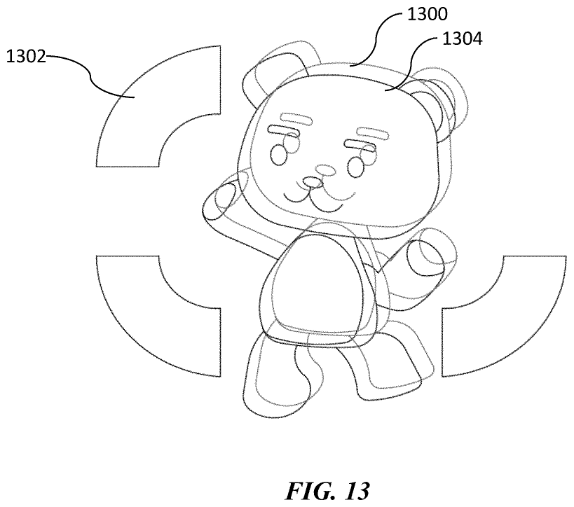

FIG. 13 is another exemplary view of the mock MRI showing an avatar 1300 as the target image in a biofeedback mini-game. The stationary avatar 1300, e.g., a teddy bear, can be a static target image displayed over the mock MRI's interior view. The motion-tracking avatar 1302 is fixed or aligned with the motion of the user's head movement. The user is requested to overlap the motion-tracking avatar 1302 with the stationary avatar 1300. Furthermore, any excessive head motion can be an indicator of the user's body motion. When the user's head or body movement has exceeded a permitted range, the mis-aligned avatars 1300 and 1302 instantaneously becomes blurred, serving as biofeedback reference to illustrate users movement, i.e., more movement leads to a more blurred image. According to some embodiment, the breaching cue 1302 can be a visual cue or an audio cue, or a combination of the two. According to some embodiments, the breaching cue 1302 can be a visual reminder, e.g., “stay still” displayed next to the selected avatar 1300. Furthermore, when the user stays still, the virtual reality headset can display compliments such as “doing great,” “almost done,” and “you are awesome!” Accordingly, the user can obtain live training to improve his/her performance in the actual MRI.

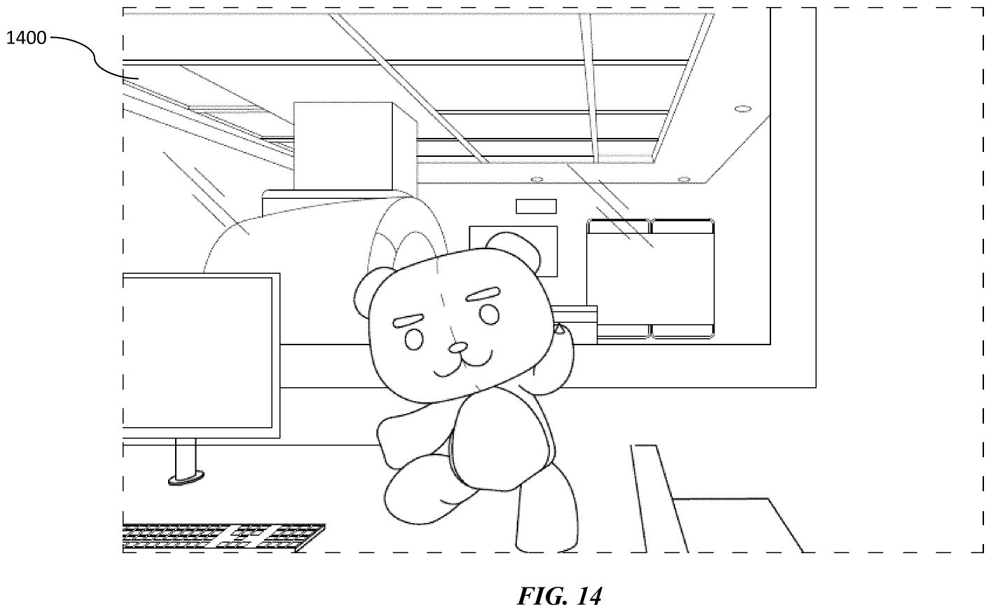

FIG. 14 is another example image of the introduction video 1400. It can show a conclusion of the mock MRI, in which the avatar congratulates the user to complete the introduction, training and/or assessment.

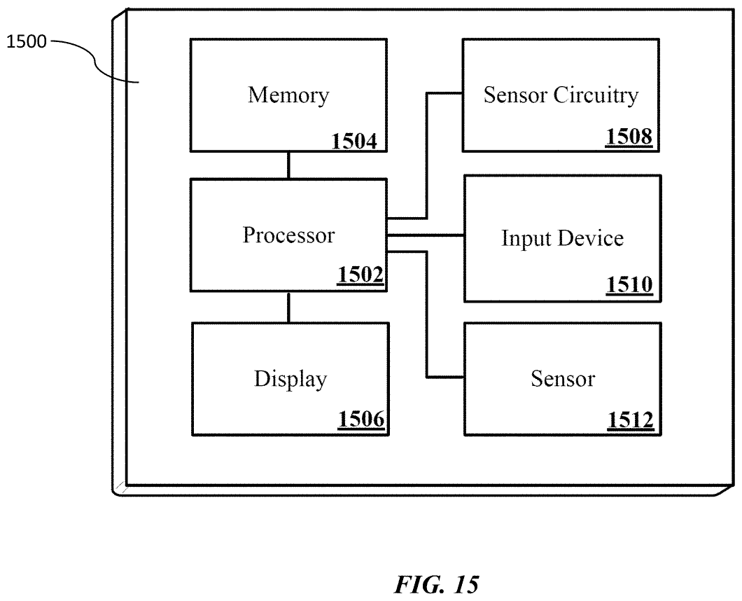

FIG. 15 is a block diagram of basic components of a virtual reality headset 1500 implementing the features and processes of FIGS. 1-14. In order to provide various functionality described herein, FIG. 15 illustrates an example set of basic components of a virtual reality headset 1500, such as the virtual reality headset described with respect to FIG. 3. In this example, the virtual reality headset 1500 includes at least one central processor 1502 for executing instructions that can be stored in at least one memory device or element 1504. As would be apparent to one of ordinary skill in the art, the virtual reality headset 1500 can include many types of memory, data storage, or computer-readable storage media, such as a first data storage for program instructions such as the predictive analytic model for execution by the processor 1502, the same or separate storage can be used for images, biosensing data, or the MRI datasets, a removable storage memory can be available for sharing information with other devices, etc. The memory device 1504 can store data, programs or instructions related to the MRI database and the predictive analytic model as described herein.

The virtual reality headset 1500 typically can include some type of display element 1506, such as a head-mounted display, although it might convey information via other means, such as through audio headphones or speakers. In at least some embodiments, the display element 1506 provides for a built-in light indicator. As described herein, the built-in light indicator can be used to show the prediction of the user's suitability for the actual MRI. In at least some embodiments, other output devices, e.g., an external display in connection with the virtual reality headset 1500, can be used to show the prediction or result of the mock MRI. In at least some embodiments, the display element 1506 provides for touch or swipe-based input using, for example, capacitive or resistive touch technology.

As discussed, the virtual reality headset 1500 in many embodiments will include at least one sensor 1512. The sensor 1512 can be, for example, biometric sensors. In at least some embodiments, the sensor 1512 includes a camera, an accelerometer, a magnetometer, a gyroscope, a blood pressure meter, a pulse sensor, a head motion sensor, a respiration sensor, a heart rate sensor, an eye-tracking sensor, an EDA sensor, an EMG sensor, an EEG sensor, an ECG sensor, and a galvanic skin response sensor. For example, the sensor 1512 can be one or more front-facing cameras that are able to image a user, people, or objects in the vicinity of the headset.

The virtual reality headset 1500 can include sensor circuitry 1508, which at least some embodiments can attempt to determine one or more sensor data values and attempt to adjust the sensor's sensitivity, gain, or other such parameters to attempt to improve the quality of subsequently captured data.

The virtual reality headset 1500 can include at least one additional input device 1510 able to receive input from a user. This input can include, for example, a controller, push-button, touchpad, touch screen, microphone, wheel, joystick, keyboard, mouse, trackball, keypad, or any other such device or element whereby a user can input a command to the headset. These I/O devices could be connected by a wireless infrared or Bluetooth or other link as well in some embodiments. In some embodiments, however, such a device might not include any buttons at all and might be controlled only through a combination of visual (e.g., gesture) and audio (e.g., spoken) commands such that a user can control the headset without having to be in contact with the device.

The virtual reality headset 1500 can include at least one network interface (not shown) able to transmit the data collected by sensor 1512. In at least some embodiments, the network interface can enable Bluetooth communication between the sensor and the headset or a computing device. Other wired or wireless communication protocols can be enabled via at least one network interface.

A number of implementations have been described. Nevertheless, it will be understood that various modifications may be made. For example, other steps may be provided, or steps may be eliminated from the described flows, and other components may be added to, or removed from, the described systems. Accordingly, other implementations are within the scope of the following claims.

Although a variety of examples and other information was used to explain aspects within the scope of the appended claims, no limitation of the claims should be implied based on particular features or arrangements in such examples, as one of ordinary skill would be able to use these examples to derive a wide variety of implementations. The described features and steps are disclosed as examples of components of systems and methods within the scope of the appended claims.

What is claimed is:

1. A method of using a virtual reality headset for a mock Magnetic Resonance Imaging (MRI), comprising:
generating, via the virtual reality headset, a simulation of an actual MRI to a user, wherein the simulation comprises the audio and visual experiences of the actual MRI;
creating biometric data of the user via collected biosensing data from one or more biometric sensors in communication with the virtual reality headset over a predetermined period of time;
assessing, by analyzing the digital biometric data in a predictive analytic model, the user's suitability for the actual MRI; and
generating a prediction of the user's suitability for the actual MRI.

2. The method of claim 1, wherein the created digital biometric data comprises one of the user's age, gender, movement, stress, alertness, and emotional valence data.

3. The method of claim 2, further comprising:
compiling one or more user scores reflecting the user's age, gender, movement, stress, alertness, and emotional valence data, wherein the one or more user scores are inputs of the predictive analytic model.

4. The method of claim 1, wherein the user's created digital biometric data comprises at least movement data, and the predetermined threshold comprises a predetermined frequency and/or degree of the movement.

5. The method of claim 1, wherein the one or more biometric sensors comprises at least one of a camera, an accelerometer, a magnetometer, a gyroscope, a blood pressure meter, a pulse sensor, a head motion sensor, a respiration sensor, a heart rate sensor, an eye-tracking sensor, an EDA sensor, an EMG sensor, an EEG sensor, an ECG sensor, and a galvanic skin response sensor.

6. The method of claim 1, further comprising:
generating the predictive analytic model based on an MRI database, wherein the prediction is generated by assessing the user's digital biometric data in relation to the predictive analytic model.

7. The method of claim 1, wherein the predictive analytic model is configured to process the digital biometric data as input parameters via one or more data processing techniques.

8. The method of claim 1, further comprising:
displaying a target image in the simulation;
determining that the user's digital biometric data exceeds at least one predetermined threshold; and
displaying, via the virtual reality headset, a breaching cue associated with the target image, wherein the breaching cue comprises at least one visual or audio reminder.

9. The method of claim 1, further comprising:
displaying, via an indicator of the virtual reality headset, the prediction of the user's suitability for the actual MRI.

10. The method of claim 1, wherein the one or more biometric sensors are configured to communicate with the virtual reality headset via a network protocol.

11. The method of claim 1, further comprising:
displaying an introduction video of the actual MRI via the virtual reality headset, wherein the introduction video includes an animated avatar.

12. A method of using a virtual reality headset for a mock Magnetic Resonance Imaging (MRI), comprising:
generating, via the virtual reality headset, a simulation of an actual MRI to a user;
displaying a target image in the simulation;
creating biometric data of the user via collected biosensing data from one or more biometric sensors in communication with the virtual reality headset over a predetermined period of time;
evaluating the digital biometric data in relation to a predictive analytic model for the user's suitability in an actual MRI;
determining that the user's digital biometric data exceeds at least one predetermined threshold; and
displaying, via the virtual reality headset, real-time biofeedback associated with the target image, wherein the real-time biofeedback comprises at least one visual or audio reminder.

13. The method of claim 12, wherein the digital biometric data comprises one or more of the user's age, gender, movement, stress, alertness, and emotional valence data.

14. The method of claim 13, further comprising:
compiling one or more user composite scores reflecting the user's age, gender, movement, stress, alertness, and emotional valence data, wherein the one or more user composite scores are inputs of the predictive analytic model.

15. The method of claim 12, wherein the one or more biometric sensors comprises at least one of a camera, an accelerometer, a magnetometer, a gyroscope, a blood pressure meter, a pulse sensor, a head motion sensor, a respiration sensor, a heart rate sensor, an eye-tracking sensor, an EDA sensor, an EMG sensor, an EEG sensor, an ECG sensor, and a galvanic skin response sensor.

16. The method of claim 12, further comprising:
generating a prediction of the user's suitability for the actual MRI; and
displaying, via an indicator of the virtual reality headset, the prediction of the user's suitability for the actual MRI.

17. The method of claim 12, wherein the predictive analytic model is configured to process the digital biometric data as input parameters via one or more data processing techniques.

18. A virtual reality headset system for a mock Magnetic Resonance Imaging (MRI), comprising:
a processor;
one or more biometric sensors; and
memory storing instructions that, when executed by the processor, cause the virtual reality headset to:
generate a simulation of an actual MRI to a user;
display a target image in the simulation;
create digital biometric data of the user via one or more biometric sensors in communication with the virtual reality headset over a predetermined period of time;
assess, by analyzing the digital biometric data in a predictive analytic model, the user's suitability for the actual MRI; and
generate a prediction of the user's suitability for the actual MRI.

19. The virtual reality headset system of claim 18, further comprising instruction that, when executed by the processor, cause the virtual reality headset to:
determine that the user's digital biometric data exceeds at least one predetermined threshold; and
display, via the virtual reality headset, a breaching cue associated with the target image, wherein the breaching cue comprises at least one visual or audio reminder.

20. The virtual reality headset system of claim 18, further comprising instruction that, when executed by the processor, cause the virtual reality headset to:
generate the predictive analytic model based on an MRI database, wherein the prediction is generated by assessing the user's digital biometrics data in relation to the predictive analytic model.
